(19) United States
(12) Patent Application Publication
(10) Pub. No.: US 2021/0363091 A1
(43) Pub. Date: Nov. 25, 2021

(54) ELECTRIC HEATING OF BOILER FEEDWATER IN THE MANUFACTURE OF PURIFIED AROMATIC CARBOXYLIC ACIDS

(71) Applicant: BP CORPORATION NORTH AMERICA INC., Houston, TX (US)

(72) Inventors: Frederick Clark, Naperville, IL (US); Thomas Bartos, Naperville, IL (US); Xiaolin Liang, Naperville, IL (US)

(21) Appl. No.: 16/635,798

(22) PCT Filed: Aug. 1, 2017
(86) PCT No.: PCT/CN2017/095405

§ 371 Date: Jan. 31, 2020

(51) Int. Cl. C07C 51/265; C07C 51/42

(52) U.S. Cl. CPC C07C 51/265; C07C 51/42

(57) ABSTRACT

Processes for manufacturing purified aromatic carboxylic carboxylic acids includes: generating high-pressure steam (402) from boiler feed water supplied to a boiler (404); heating a crude aromatic carboxylic acid using the high-pressure steam (402), whereby the high pressure steam (402) is condensed to form a high-pressure condensate (426); and purifying the crude aromatic carboxylic acid to form a purified aromatic carboxylic acid. The boiler feed water includes at least a portion of the high-pressure condensate (426) and makeup boiler feed water from at least one additional source. The recycled high-pressure condensate (426) is pre-heated with an electric heater (480) using electricity generated in an off-gas treatment zone (350).

### TECHNICAL FIELD

The present teachings relate generally to processes for manufacturing purified aromatic carboxylic acids, and in particular, to processes for electric heating of boiler feedwater in the manufacture of aromatic carboxylic acids prior to purification.

### BACKGROUND

Terephthalic acid (TA) and other aromatic carboxylic acids may be used in the manufacture of polyesters (e.g., via their reaction with ethylene glycol and/or higher alkylene glycols). Polyesters in turn may be used to make fibers, films, containers, bottles, other packaging materials, molded articles, and the like.

In commercial practice, aromatic carboxylic acids have been made by liquid phase oxidation of methyl-substituted benzene and naphthalene feedstocks in an aqueous acetic acid solvent. The positions of the methyl substituents correspond to the positions of carboxyl groups in the aromatic carboxylic acid product. Air or other sources of oxygen (e.g., typically in a gaseous state) have been used as oxidants in the presence, for example, of a bromine-promoted catalyst that contains cobalt and manganese. The oxidation is exothermic and yields aromatic carboxylic acid together with by-products, including partial or intermediate oxidation products of the aromatic feedstock, and acetic acid reaction products (e.g., methanol, methyl acetate, and methyl bromide). Water is also generated as a by-product.

Pure forms of aromatic carboxylic acids are oftentimes desirable for the manufacture of polyesters to be used in important applications (e.g., fibers and bottles). Impurities in the acids (e.g., by-products generated from oxidation of aromatic feedstocks and, more generally, various carbonyl-substituted aromatic species) are thought to cause and/or correlate with color formation in polyesters made therefrom, which in turn leads to off-color in polyester converted products. Aromatic carboxylic acids having reduced levels of impurities may be made by further oxidizing crude products from liquid phase oxidation as described above at one or more progressively lower temperatures and oxygen levels. In addition, partial oxidation products may be recovered during crystallization and converted into the desired acid product.

Pure forms of terephthalic acid and other aromatic carboxylic acids having reduced amounts of impurities—for example, purified terephthalic acid (PTA)—have been made by catalytically hydrogenating less pure forms of the acids or so-called medium purity products in solution at elevated temperature and pressure using a noble metal catalyst. Less pure forms of the acids may include crude product that contains aromatic carboxylic acid and by-products from liquid phase oxidation of the aromatic feedstock. In commercial practice, liquid phase oxidation of alkyl aromatic feed materials to crude aromatic carboxylic acid, and purification of the crude product, are oftentimes conducted in continuous integrated processes in which crude product from the liquid phase oxidation is used as a starting material for the purification.

Purification of crude aromatic carboxylic acid has been accomplished through hydrogenation. Crude aromatic carboxylic acid is usually pre-heated prior to being fed to the hydrogenation reactor, which typically operates at a temperature of about 260° C. to about 290° C. One manner in which such pre-heating is accomplished is through indirect heat exchange with high pressure steam. The high pressure steam is condensed during heat exchange, and the resulting condensate may be let down to form low pressure condensate and low pressure steam which may be used in other process steps. In the alternative, the high pressure condensate may be recycled as feed water to the boiler used to generate the steam.

The fuel costs associated with generation of the high pressure steam contributes to the overall variable costs of the process for manufacturing the purified aromatic carboxylic acid. There continues to be a desire to reduce such variable costs through more efficient energy management strategies.

### SUMMARY

The scope of the present invention is defined solely by the appended claims, and is not affected to any degree by the statements within this summary.

By way of introduction, one embodiment of a process for manufacturing purified aromatic carboxylic acid in accordance with the present teachings comprises: directing a feed comprising a substituted aromatic hydrocarbon with gaseous oxygen in a liquid phase oxidation reaction mixture comprising an acetic acid solvent and water and in the presence of a catalyst composition comprising at least one heavy metal component in a reaction zone at temperature and pressure effective to maintain a liquid phase oxidation reaction mixture and form a crude aromatic carboxylic acid, and a high pressure vapor phase comprising acetic acid, water and minor amounts of the feed, the crude aromatic carboxylic acid and by-products; separating the high pressure vapor phase to form an acetic acid-rich, water-lean liquid and a high pressure off-gas comprising water vapor; treating the high pressure off-gas in an off-gas treatment zone; generating electricity from the high pressure off-gas; generating high-pressure steam from boiler feed water supplied to a boiler; wherein at least a portion of the boiler feed water is pre-heated with an electric heater using the electricity generated from the high pressure off-gas; heating the crude aromatic carboxylic acid in a heating zone using the high-pressure steam, whereby the high pressure steam is condensed in the heating zone to form a high-pressure condensate; and purifying the crude aromatic carboxylic acid to form a purified aromatic carboxylic acid; wherein the boiler feed water comprises at least a portion of the high-pressure condensate.

Other aspects of the present invention will be apparent in view of the description that follows.

### BRIEF DESCRIPTION OF THE DRAWINGS

FIG. 1 shows a process flow diagram for the manufacture of purified forms of aromatic carboxylic acids in accordance with one embodiment of the present invention.

### DETAILED DESCRIPTION

By way of general introduction, the present invention is directed to processes for manufacturing purified aromatic carboxylic acids using efficient heat exchange configurations in the pre-heating of crude aromatic carboxylic acids prior to purification. High-pressure steam is used to heat crude aromatic carboxylic acid in a pre-heating zone prior to the purification of the crude aromatic carboxylic acid. At least a portion of high-pressure condensate generated from the condensation of the high pressure steam in the pre-heating zone may be recycled to provide at least a portion of boiler feed water from which the high pressure steam is generated. The high-pressure condensate is heated with an electric heater prior to being recycled to the boiler. The process reduces boiler furnace load, resulting in fuel savings and lower carbon footprint compared to prior art systems.

According to a first process for manufacturing a purified aromatic carboxylic acid in accordance with the present teachings comprises reacting a feed comprising a substituted aromatic hydrocarbon with gaseous oxygen in a liquid phase oxidation reaction mixture comprising an acetic acid solvent and water and in the presence of a catalyst composition comprising at least one heavy metal component in a reaction zone at temperature and pressure effective to maintain a liquid phase oxidation reaction mixture and form a crude aromatic carboxylic acid, and a high pressure vapor phase comprising acetic acid, water and minor amounts of the feed, the crude aromatic carboxylic acid and by-products; separating the high pressure vapor phase to form an acetic acid-rich, water-lean liquid and a high pressure off-gas comprising water vapor; treating the high pressure off-gas in an off-gas treatment zone; generating electricity from the high pressure off-gas in the off-gas treatment zone; generating high-pressure steam from boiler feed water supplied to a boiler; wherein at least a portion of the boiler feed water is pre-heated with an electric heater using the electricity generated from the high pressure off-gas; heating the crude aromatic carboxylic acid in a heating zone using the high-pressure steam, whereby the high pressure steam is condensed in the heating zone to form a high-pressure condensate; and purifying the crude aromatic carboxylic acid to form a purified aromatic carboxylic acid; wherein the boiler feed water comprises at least a portion of the high-pressure condensate.

In some embodiments, the boiler feed water also includes makeup boiler feed water from an additional source, such as water that has been de-aerated using low-pressure steam. In some embodiments, the high-pressure condensate and the makeup boiler feed water are combined prior to delivery of the boiler feed water to the boiler. In other embodiments, the high-pressure condensate and the makeup boiler feed water are combined in situ in the boiler, for example, in the boiler's steam drum. In some embodiments, the makeup boiler feed water is at a lower temperature than of the high-pressure condensate at least prior to their combination. In some embodiments, the high-pressure condensate has a temperature of between about 250° C. and about 305° C. which is delivered to the boiler at a pressure of between about 80 bar(g) and about 120 bar(g). In some embodiments, the makeup boiler feed water has a temperature of between about 100° C. and about 150° C., which is delivered to the boiler at a pressure of between about 80 bar(g) and about 120 bar(g).

Additional features of the above-described processes for manufacturing purified forms of aromatic carboxylic acid in accordance with the present teachings will now be described in reference to the drawing figures.

Processes for manufacturing purified aromatic carboxylic acids from substituted aromatic hydrocarbons, along with ancillary processes for recovering energy and purifying waste streams are generally known in the art and more fully described, for example, in US. Pat. Nos. 5,723,656, 6,137,001, 7,935,844, 7,935,845, 8,173,834, and 9,315,441.

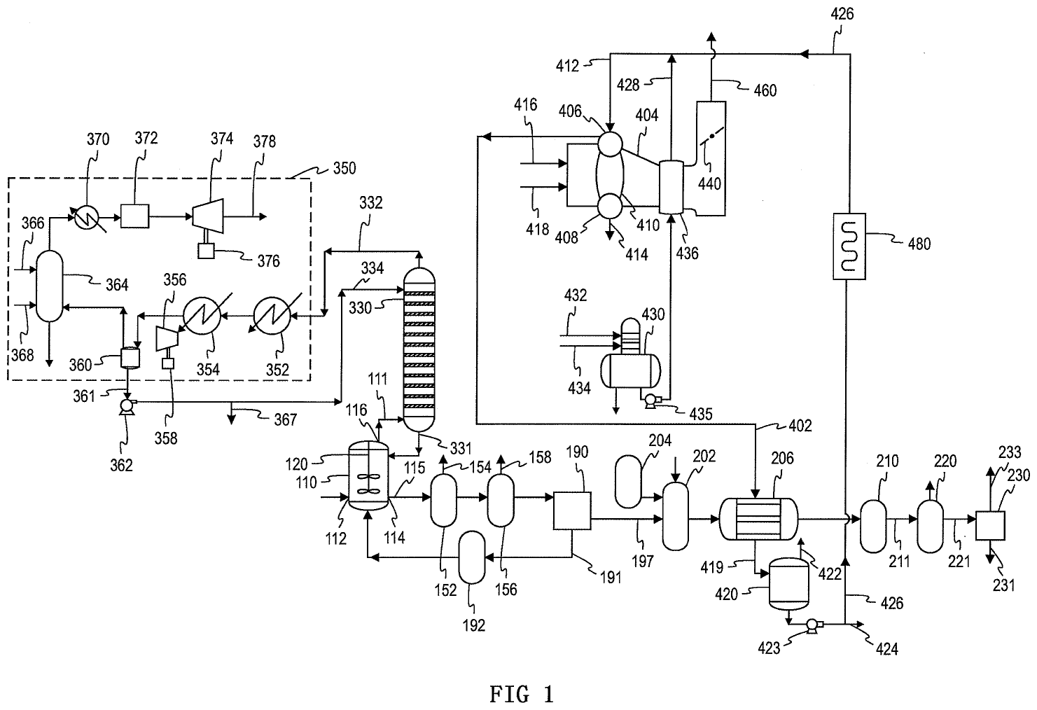

FIG. 1 shows a simplified process flow diagram for manufacturing purified forms of aromatic carboxylic acids in accordance with the present invention. Liquid and gaseous streams and materials used in the process represented in FIG. 1 may be directed and transferred through suitable transfer lines, conduits, and piping constructed, for example, from materials appropriate for process use and safety. It will be understood that particular elements may be physically juxtaposed and, where appropriate, may have flexible regions, rigid regions, or a combination of both. In directing streams or compounds, intervening apparatuses and/or optional treatments may be included. By way of example, pumps, valves, manifolds, gas and liquid flow meters and distributors, sampling and sensing devices, and other equipment (e.g., for monitoring, controlling, adjusting, and/or diverting pressures, flows and other operating parameters) may be present.

In a representative embodiment, such as may be implemented as shown in FIG. 1, liquid feed material comprising, by way of example, at least about 99 wt. % of a substituted aromatic hydrocarbon feed material, a monocarboxylic acid solvent, an oxidation catalyst, a catalyst promoter, and air are continuously charged to oxidation reaction vessel 110 through inlets, such as inlet 112. In some embodiments, vessel 110 is a pressure-rated, continuous-stirred tank reactor.

In some embodiments, stirring may be provided by rotation of an agitator 120, the shaft of which is driven by an external power source (not shown). Impellers mounted on the shaft and located within the liquid body are configured to provide forces for mixing liquids and dispersing gases within the liquid body, thereby avoiding settling of solids in the lower regions of the liquid body.

Suitable aromatic feed materials for the oxidation generally comprise an aromatic hydrocarbon substituted at one or more positions, normally corresponding to the positions of the carboxylic acid groups of the aromatic carboxylic acid being prepared, with at least one group that is oxidizable to a carboxylic acid group. The oxidizable substituent or substituents can be alkyl groups, such as a methyl, ethyl or isopropyl groups, or groups already containing oxygen, such as a hydroxyalkyl, formyl or keto group. The substituents can be the same or different. The aromatic portion of feedstock compounds can be a benzene nucleus or it can be bi- or polycyclic, such as a naphthalene nucleus. Examples of useful feed compounds, which can be used alone or in combinations, include toluene, ethylbenzene and other alkyl-substituted benzenes, o-xylene, p-xylene, m-xylene, tolualdehydes, toluic acids, alkyl benzyl alcohols, 1-formyl-4-methylbenzene, 1-hydroxymethyl-4-methylbenzene, methylacetophenone, 1,2,4-trimethylbenzene, 1-formyl-2,4-dimethyl-benzene, 1,2,4,5-tetramethyl-benzene, alkyl-, formyl-, acyl-, and hydroxylmethyl-substituted naphthalenes, such as 2,6-diethylnaphthalene, 2,6-diethylnaphalene, 2,7-dimethylnaphthalene, 2,7-diethylnaphthalene, 2-formyl-6-methylnaphthalene, 2-acyl-6-methylnaphthalene, 2-methyl-6-ethylnaphthalene and partially oxidized derivatives of the foregoing.

For manufacture of aromatic carboxylic acids by oxidation of their correspondingly substituted aromatic hydrocarbon pre-cursors, e.g., manufacture of benzoic acid from mono-substituted benzenes, terephthalic acid from para-disubstituted benzenes, phthalic acid from ortho-disubstituted benzenes, and 2,6 or 2,7 naphthalene dicarboxylic acids from, respectively, 2,6- and 2,7-disubstituted naphthalenes, it is preferred to use relatively pure feed materials, and more preferably, feed materials in which content of the pre-cursor corresponding to the desired acid is at least about 95 wt. %, and more preferably at least 98 wt. % or even higher. In one embodiment, the aromatic hydrocarbon feed for use to manufacture terephthalic acid comprises para-xylene.

Solvent for the liquid phase reaction of aromatic feed material to aromatic carboxylic acid product in the liquid phase oxidation step comprises a low molecular weight monocarboxylic acid, which is preferably a C₁-C₈ monocarboxylic acid, for example acetic acid, propionic acid, butyric acid, valeric acid and benzoic acid.

Catalysts used for the liquid oxidation comprise materials that are effective to catalyze oxidation of the aromatic feed material to aromatic carboxylic acid. Preferred catalysts are soluble in the liquid phase reaction mixture used for oxidation because soluble catalysts promote contact among catalyst, oxygen gas and liquid feed materials; however, heterogeneous catalyst or catalyst components may also be used. Typically, the catalyst comprises at least one heavy metal component. Examples of suitable heavy metals include cobalt, manganese, vanadium, molybdenum; chromium, iron, nickel, zirconium, cerium or a lanthanide metal such as hafnium. Suitable forms of these metals include, for example, acetates, hydroxides, and carbonates. Preferred catalysts comprise cobalt, manganese, combinations thereof and combinations with one or more other metals and particularly hafnium, cerium and zirconium.

In preferred embodiments, catalyst compositions for liquid phase oxidation also comprise a promoter, which promotes oxidation activity of the catalyst metal, preferably without generation of undesirable types or levels of by-products. Promoters that are soluble in the liquid reaction mixture used in oxidation pre preferred for promoting contact among catalyst, promoter and reactants. Halogen compounds are commonly used as a promoter, for example hydrogen halides, sodium halides, potassium halides, ammonium halides, halogen-substituted hydrocarbons, halogen-substituted carboxylic acids and other halogenated compounds. Preferred promoters comprise at least one bromine source. Suitable bromine sources include bromo-anthracenes, Br2, HBr, NaBr, KBr, NH4Br, benzyl-bromide, bromo acetic acid, dibromo acetic acid, tetrabromoethane, ethylene dibromide, bromoacetyl bromide and combinations thereof. Other suitable promoters include aldehydes and ketones such as acetaldehyde and methyl ethyl ketone.

Reactants for the liquid phase reaction of the oxidation step also include a gas comprising molecular oxygen. Air is conveniently used as a source of oxygen gas. Oxygen-enriched air, pure oxygen and other gaseous mixtures comprising molecular oxygen, typically at levels of at least about 10 vol. %, also are useful.

The substituted aromatic hydrocarbon is oxidized in reactor 110, to form a crude aromatic carboxylic acid and by-products. In one embodiment, for example, paraxylene is converted to terephthalic acid and by-products that may form in addition to terephthalic acid include partial and intermediate oxidation products (e.g., 4-carboxybenzaldehyde, 1,4-hydroxymethyl benzoic acid, p-toluic acid, benzoic acid, and the like, and combinations thereof). Since the oxidation reaction is exothermic, heat generated by the reaction may cause boiling of the liquid phase reaction mixture and formation of an overhead vapor phase that comprises vaporized acetic acid, water vapor, gaseous by-products from the oxidation reaction, carbon oxides, nitrogen from the air charged to the reaction, unreacted oxygen, and the like, and combinations thereof.

The overhead vapor is removed from the reactor 110 through vent 116 and sent in a stream 111 to a separation zone, which in the embodiment shown is high-pressure distillation column 330. The separation zone is configured to separate water from the solvent monocarbxylic acid and return a solvent-rich liquid phase to the reactor via line 331. A water rich gas phase is removed from the separation zone via line 332 and is further processed in off-gas treatment zone 350. Reflux 334 is returned to the column 330. Examples of further processing of the overhead gas stream and reflux options for the column 330 are more fully described in US. Pat. Nos. 5,723,656, 6,137,001, 7,935,844, 7,935,845, and 8,173,834. Liquid effluent comprising solid crude aromatic carboxylic acid product is slurried in the liquid phase reaction mixture and is removed from reaction vessel 110 through slurry outlet 114 and directed in stream 115 to a crystallization zone for recovery of a solid product.

In the embodiment of the invention illustrated in FIG. 1, the crystallization zone includes multiple stirred crystallization vessels, 152 and 156 in series and in flow communication for transfer of product slurry from vessel 152 to vessel 156. Cooling in the crystallization vessels is accomplished by pressure release, with the slurry cooled in vessel 152 to a temperature in the range of about 150-190° C. and then further to about 110-150° C. in vessel 156. One or more of the crystallization vessels is vented, as at 154 and 158, respectively, for removal to heat exchange means (not shown) of vapor resulting from pressure let down and generation of steam from the flashed vapor. Vapor removed from one or more upstream crystallization vessels, such as vessel 152, to heat exchange means is preferably condensed and liquid condensate comprising water, acetic acid solvent and soluble products and by-products of the oxidation can directed to one or more downstream crystallization vessels, as at 156, to allow for recovery of crystallizable components such as crude aromatic carboxylic acid and oxidation by-products entering and condensed from the flashed vapors from one or more upstream vessel.

Crystallization vessel 156 is in fluid communication with a solid-liquid separation device 190, which is adapted to receive from the crystallization vessel a slurry of solid product comprising the crude aromatic carboxylic acid and oxidation by-products in a mother liquor from the oxidation comprising monocarboxylic acid solvent and water, and to separate a crude solid product comprising terephthalic acid and by-products from the liquid. Separation device 190 is a centrifuge, rotary vacuum filter or pressure filter. In preferred embodiments of the invention, the separation device is a pressure filter adapted for solvent exchange by positive displacement under pressure of mother liquor in a filter cake with wash liquid comprising water. The oxidation mother liquor that results from the separation exits separation device 190 in stream 191 for transfer to mother liquor drum 192. A major portion of the mother liquor is transferred from drum 192 to oxidation reactor 110 for return to the liquid phase oxidation reaction of acetic acid, water, catalyst and oxidation reaction by-products dissolved or present as fine solid particles in the mother liquor. Crude solid product and impurities comprising oxidation by-products of the feedstock is conveyed, with or without intermediate drying and storage, from separation device 190 to purification solution make up vessel 202 in stream 197. The crude solid product is slurried in make up vessel 202 in purification reaction solvent, all or at least a portion, and preferably about 60 to about 100 wt. %, of which, comprises a second liquid phase from an off-gas separation of water and acetic acid in a vapor phase removed from reactor 110 to column 330 and by-products of the oxidation. If used, make up solvent, such as fresh demineralized water or suitable recycle streams such as liquid condensed from vapors resulting from pressure letdown in crystallization of purified terephthalic acid product as discussed below, can be directed to make up tank 202 from vessel 204. Slurry temperature in the make up tank preferably is about 80 to about 100° C.

Crude aromatic carboxylic acid product is dissolved to form a purification reaction solution by heating, for example to about 260 to about 290° C. in makeup tank 202 and by passage through a heating zone comprising one or more heat exchangers 206 as it is transferred to purification reactor 210. In reactor 210, the purification reaction solution is contacted with hydrogen under pressure preferably ranging from about 85 to about 95 bar (g) in the presence of a hydrogenation catalyst.

Catalysts suitable for use in purification hydrogenation reactions comprise one or more metals having catalytic activity for hydrogenation of impurities in impure aromatic carboxylic acid products, such as oxidation intermediates and by-products and/or aromatic carbonyl species. The catalyst metal preferably is supported or carried on a support material that is insoluble in water and unreactive with aromatic carboxylic acids under purification process conditions. Suitable catalyst metals are the Group VIII metals of the Periodic Table of Elements (IUPAC version), including palladium, platinum, rhodium, osmium, ruthenium, iridium, and combinations thereof. Palladium or combinations of such metals that include palladium are most preferred. Carbons and charcoals with surface areas of several hundreds or thousands m²/g surface area and sufficient strength and attrition resistance for prolonged use under operating conditions are preferred supports. Metal loadings are not critical but practically preferred loadings are about 0.1 wt % to about 5 wt % based on total weight of the support and catalyst metal or metals. Preferred catalysts for conversion of impurities present in impure aromatic carboxylic acid products contain about 0.1 to about 3 wt % and more preferably about 0.2 to about 1 wt % hydrogenation metal. In one particular embodiment, the metal comprises palladium.

A portion of the purification liquid reaction mixture is continuously removed from hydrogenation reactor 210 in stream 211 to crystallization vessel 220 where purified aromatic carboxylic acid product and reduced levels of impurities are crystallized from the reaction mixture by reducing pressure on the liquid. The resulting slurry of purified aromatic carboxylic acid and liquid formed in vessel 220 is directed to solid-liquid separation apparatus 230 in stream line 221. Vapors resulting from pressure letdown in the crystallization can be condensed by passage to heat exchangers (not shown) for cooling and the resulting condensed liquid redirected to the process, for example as recycle to purification feed makeup tank 202, through suitable transfer lines (not shown). Purified aromatic carboxylic acid product exits solid-liquid separation device 230 in stream 231. The solid-liquid separation device can be a centrifuge, rotary vacuum filter, a pressure filter or combinations of one or more thereof.

Purification mother liquor from which the solid purified aromatic carboxylic acid product is separated in solid-liquid separator 230 comprises water, minor amounts of dissolved and suspended aromatic carboxylic acid product and impurities including hydrogenated oxidation by-products dissolved or suspended in the mother liquor. Purification mother liquor directed in stream 233 may be sent to waste water treatment facilities or alternatively may be used as reflux 334 to the column 330, as more fully described, for example, in U.S. Pat. Nos. 5,723,656, 6,137,001, 7,935,844, 7,935,845, and 8,173,834.

As discussed above, crude aromatic carboxylic acid product is heated in a heating zone having heat exchanger 206. Those skilled in the art appreciate that although one heat exchanger is shown, the heating zone may include multiple heat exchangers including pre-heaters upstream of heat exchanger 206. In one embodiment, the heat exchanger is a tube and shell exchanger in which the crude aromatic carboxylic acid is heated by indirect contact heating with high pressure steam supplied by line 402.

The high pressure steam 402 is generated by a boiler 404. In one embodiment, the boiler 404 is a standard type-D Nebraska boiler available from Cleaver-Brooks of Lincoln, Nebraska. The boiler 404 includes a steam drum 406 and a mud drum 408 connected by a plurality of riser and downcomer tubes 410. Boiler feed water is introduced into the steam drum 406 through line 412. The boiler feed water is delivered as a liquid at pressures slightly exceeding the pressure of the steam drum 406 and at temperatures which are sub-cooled relative to the delivery pressure. The density of the boiler feed water entering the steam drum 406 is greater compared with the density of the two-phase liquid-vapor water mixture in the steam drum 406. This density gradient thereby promotes a thermosiphon effect as the entering, higher density liquid flows downward through the downcomer tubes 410 and into the lower mud drum 408 which, in turn, forces lower density, two-phase water mixtures to flow upward in the riser tubes 410 from the mud drum 408 into the steam drum 406. High pressure steam is removed from the steam drum 406 through line 402. Bottom blowdown, comprising water with impurities, is removed from the mud drum 408 through line 414 at a rate of about 1% to 3% of the boiler feed water 412 entering the steam drum to avoid the build-up of corrosive materials. A fuel, such as natural gas, is injected through line 416 and a source of oxygen, such as air, is introduced through line 418 for providing the combustion heat source (not shown) for the boiler 404. Flue gas 460 is removed from the stack and is controlled with a damper 440.

The boiler feed water 412 includes at least a portion of the high pressure condensate 419 that is formed by the condensation of the high pressure steam 402 in the shell side of the heat exchanger 206. In one embodiment, the high pressure condensate 419 exiting the heat exchanger 206 is introduced into flash drum 420, which is maintained at pressure close to condensate 419 to remove remaining steam through line 422. The steam 422 may be used in other parts of the process (not shown). A portion of the high pressure condensate may be withdrawn through line 424 to be used in other parts of the process, however, at least a portion of the high pressure condensate exiting the flash drum 420 is sub-cooled and further pressurized by pump 423 before being recycled to be used as boiler feed water 412. In one embodiment, at least 65 wt%, or up to at least 97 wt%, of the high pressure condensate that is generated in the heat exchanger 206 is recycled for use as boiler feed water 412.

In one embodiment, the makeup boiler feed water is at a lower temperature than the high-pressure condensate prior their combination. In one embodiment, the high-pressure condensate has a temperature of between about 250 ° C. and about 305 ° C. which is delivered to the boiler at a pressure of between about 80 bar(g) and about 120 bar(g). In one embodiments, the makeup boiler feed water has a temperature of between about 100 ° C. and about 150 ° C., which is delivered to the boiler at a pressure of between about 80 bar(g) and about 120 bar(g).

In one embodiment, the boiler feed water 412 also includes water from at least one other source. In the embodiment shown in FIG. 1, the boiler water feed 412 includes the recycled high pressure condensate 426 and make-up feed water 428 supplied from tray type deaerator 430 and pressurized by pump 435. Deionized water is introduced into the deaerator 430 through line 432 and low-pressure steam is introduced into deaerator 430 through line 434. The deaerator 430 removes dissolved oxygen and other dissolved gases from the make-up feed water 428. In the embodiment shown, the make-up feed water 428 is combined with the recycled high pressure condensate 426 prior to the introduction of the boiler feed water 412 into the boiler 404.

The recycled high pressure condensate 426 is preheated prior to being feed to boiler 404. In the embodiment shown in FIG. 1, the recycled high pressure condensate 426 is heated with an electric heater 480. In some embodiments, the power to the electric heater is generated in an off-gas treatment zone 350 as described below. Pre-heating the recycled high pressure condensate lowers the load on the boiler furnace and reduces the carbon footprint of the overall process for manufacturing the aromatic carboxylic acid.

Reaction off-gas generated by the liquid phase oxidation of para-xylene feedstock in reactor vessel 110 is removed from the reactor through vent 116 and directed in stream 111 to separation device 330 which, in the embodiments represented in FIG. 1, is a high pressure distillation column having a plurality of trays preferably providing about 30 to about 50 theoretical plates. The vapor stream from oxidation is introduced to column 330 preferably at temperature and under pressure of about 150 to about 225° C. and about 4 to about 21 kg/cm² gauge, respectively, and not substantially less than in oxidation reactor 110. A reflux stream 334 returns fluids to the column. In the embodiment shown, the reflux fluid comprises liquid condensate recovered from the off-gas-treatment zone 350.

Water and solvent acetic acid vapors in the high pressure vapor phase introduced to the distillation column are separated, such that an acetic acid-rich, water-lean liquid phase and a separator exit gas under pressure are formed. At least 95 wt. % of the acetic acid in the high pressure vapor from oxidation is separated into the liquid phase. The liquid phase preferably comprises about 60 to about 85 wt % acetic acid and preferably no more than about 25 wt. % water. It also comprises minor amounts of other components less volatile than the acetic acid, such as terephthalic acid and para-xylene oxidation by-products such as p-toluic acid and benzoic acid introduced with the purification mother liquor reflux and may also include other components such as solvent by-products from oxidation. A high pressure gas from the separation primarily comprises water vapor and also contains unreacted oxygen gas, minor amounts of solvent acetic acid vapors, unreacted para-xylene, oxidation by-products, carbon oxides, and nitrogen introduced from the air used as the oxygen source for oxidation.

Liquid phase resulting from separation in distillation column 330 exits the column at a lower portion thereof and preferably is returned directly or indirectly to oxidation reactor 110, as in stream 331. Return of the liquid phase to oxidation provides make up solvent acetic acid to the oxidation reaction and can reduce feedstock loss by allowing for conversion to desired products of by-products condensed from the oxidation vapor phase as well as those recycled from purification mother liquor reflux to the column.

High pressure gas resulting from separation of water and acetic acid vapors in distillation column 330 is removed from the column and directed to off-gas treatment zone 350. The high pressure gas is condensed in one or more condensers 352 and 354. Preferably, condensation is conducted such that liquid condensate water at a temperature of about 40 to about 60° C. is recovered in at least one stage. In the embodiment illustrated in the figure, condensation is conducted by indirect heat exchange in condensing means 352 with water at a temperature of about 120 to about 170° C., with effluent from the condenser 352 directed to condenser 354 for further condensation using cooling water at about 30 to about 40° C. Gas and liquid effluent from condenser 354 is directed to drum 360 in which condensate liquid comprising water is collected and removed in stream 361 and from which a condenser exhaust gas under pressure is withdrawn and directed to absorber 364. Condensate liquid recovered from the pressurized exit gas from distillation column 330 by condensation in condensors 352 and 354 is at least about 95 wt. % water and also comprises minor amount of organic impurities. The condensate liquid is transferred in stream 367 to one or more vessels or liquid receptacles used in or for the purification steps. For example, a substantial portion, of the condensate liquid may be transferred to purification solution make up tank 202 for use in forming the crude product slurry and purification reaction solution that is directed to purification reactor 210. Other purification vessels and liquid receiving equipment and uses to which the condensate liquid can be directed include crystallization vessel 220 for use as clean make-up solvent to replace purification reaction liquid vaporized in the crystallizer and solid liquid separation device 230 for use as wash liquid or seal flush. The condensate liquid also is suitable for uses outside purification, such as reflux to distillation column 330 and wash liquid for solvent exchange “filters” used for separating solid products recovered from oxidation from oxidation mother liquor.

Water used as heat exchange fluid for condensation of pressurized gas from distillation column 330 is heated by heat exchange in condenser 354 to form pressurized steam which can be directed to an energy recovery device such as expander 356 and generator 358. In one embodiment, the electricity generated in generator 358 is used to power the electric heater 480 for the high pressure condensate recycle.

Uncondensed exhaust gas from condensation removed from drum 360 comprises incondensable components such as unconsumed oxygen from oxidation, nitrogen from the air used as oxygen source to the oxidation, carbon oxides from such air as well as from reactions in oxidation, and traces of unreacted para-xylene and its oxidation by-products, methyl acetate and methanol, and methyl bromide formed from the bromine promoter used in oxidation. In the embodiment illustrated in the figure, the uncondensed gas is substantially free of water vapor owing to substantially complete condensation into the condensate liquid recovered in the condensing means.

Uncondensed exhaust gas exiting drum 360 is under pressure of about 10 to about 15 kg/cm² and can be transferred directly to a power recovery device or to a pollution control device for removing corrosive and combustible species in advance of power recovery. As depicted in FIG. 1, uncondensed gas is first directed to treatment to remove unreacted feed materials and traces of solvent acetic acid and/or reaction products thereof remaining in the gas. Thus, uncondensed gas exiting drum 360 is directed to high pressure absorber 364 for stripping para-xylene, acetic acid, methanol and methyl acetate without substantial loss of pressure. Absorption vessel 364 is adapted for receipt of the substantially water-depleted gas remaining after condensation and for separation of para-xylene, solvent acetic acid and its reaction products from oxidation from the gas by contact with one or more liquid scrubbing agents. Inlets for addition of scrubbing agent to the absorber in streams 366 and 368, respectively, are disposed at one or more upper, and one or more lower portions of the absorber vessel 364. The absorber 364 also includes an upper vent from which a scrubbed gas under pressure comprising incondensable components of the inlet gas to the absorber is removed and a lower outlet for removal of a liquid acetic acid stream into which components from the gas phase comprising one or more of para-xylene, acetic acid, methanol and/or methyl acetate have been scrubbed. A bottoms liquid is removed from a lower portion of the tower and can be directed to reactor 110 for reuse of recovered components.

Pressurized gas removed from the vent from the high pressure absorber, can be directed to pollution control means for conversion of organic components and carbon monoxide in the pressurized gas from the condenser or the absorber to carbon dioxides and water. A preferred pollution control means is a catalytic oxidation unit adapted for receiving the second pressurized gas, optionally heating it to promote combustion and directing the gas into contact with a high temperature-stable oxidation catalyst disposed on a cellular or other substantially porous support such that gas flow through the device is substantially unaffected. In the embodiment shown, overhead gas from absorber 364 is directed to pollution control system which includes preheater 370 and catalytic oxidation unit 372. The gas is heated to about 250 to 450° C. in the preheater and passed under pressure of about 10 to 15 kg/cm² to catalytic oxidation unit 372 where organic components and by-products are oxidized to compounds more suited for beneficial environmental management.

An oxidized high pressure gas is directed from catalytic oxidation unit 372 to expander 374 which is connected to generator 376. Energy from the oxidized high pressure gas is converted to work in the expander 374 and such work is converted to electrical energy by generator 376. At least a portion of the electricity generated by generator 376 may be used to power the electric heater 480 to pre-heat the recycled condensate. Expanded gas exits the expander and can be released to the atmosphere by stream 378, preferably after caustic scrubbing and/or other treatments for appropriately managing such releases.

The entire contents of each and every patent and non-patent publication cited herein are hereby incorporated by reference, except that in the event of any inconsistent disclosure or definition from the present specification, the disclosure or definition herein shall be deemed to prevail.

The foregoing detailed description and the accompanying drawings have been provided by way of explanation and illustration, and are not intended to limit the scope of the appended claims. Many variations in the presently preferred embodiments illustrated herein will be apparent to one of ordinary skill in the art, and remain within the scope of the appended claims and their equivalents.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding claim—whether independent or dependent—and that such new combinations are to be understood as forming a part of the present specification.

1. A process for manufacturing a purified aromatic carboxylic acid comprising:
reacting a feed comprising a substituted aromatic hydrocarbon with a gas comprising O₂ in a liquid phase oxidation reaction mixture comprising a monocarboxylic acid solvent and water and in the presence of a catalyst composition comprising at least one heavy metal component in a reaction zone at a temperature and pressure effective to maintain a liquid phase oxidation reaction mixture and form a crude aromatic carboxylic acid, and a high pressure vapor phase comprising acetic acid, water and minor amounts of the feed, the crude aromatic carboxylic acid and by-products;
separating the high pressure vapor phase to form a monocarboxylic acid-rich, water-lean liquid and a high pressure off-gas comprising water vapor;
treating the high pressure off-gas in an off-gas treatment zone;
generating electricity in the off-gas treatment zone;
generating high-pressure steam from boiler feed water supplied to a boiler; wherein at least a portion of the boiler feed water is pre-heated with an electric heater using the electricity generated in the off-gas treatment zone;
heating the crude aromatic carboxylic acid in a heating zone using the high-pressure steam, whereby the high pressure steam is condensed in the heating zone to form a high-pressure condensate; and
purifying the crude aromatic carboxylic acid to form a purified aromatic carboxylic acid;
wherein the portion of the boiler feed water that is pre-heated comprises at least a portion of the high-pressure condensate.

2. The process of claim 1, wherein generating electricity in the off-gas treatment zone comprises generating electricity from the expansion of at least a portion of the high pressure off-gas in the off-gas treatment zone.

3. The process of claim 1, wherein generating electricity in the off-gas treatment zone comprises generating electricity from the expansion of steam generated from the condensation of at least a portion of the high pressure off-gas in the off-gas treatment zone.

4. The process of claim 1 wherein the boiler feed water further comprises makeup water from at least one additional source.

5. The process of claim 4, wherein the high-pressure condensate and the makeup boiler feed water are combined prior to delivery of the boiler feed water to the boiler.

6. The process of claim 1 wherein the aromatic carboxylic acid comprises terephthalic acid.

7. The process of claim 1 wherein the monocarboxylic acid solvent is acetic acid.
